(19) United States
(12) Patent Application Publication
(10) Pub. No.: US 2026/0160692 A1
(43) Pub. Date: Jun. 11, 2026

(54) MEASUREMENT DEVICE FOR AND METHOD OF MEASURING HYDROGEN CONCENTRATION INCLUDED IN A GAS

(71) Applicant: Endress+Hauser Optical Analysis, Inc., Ann Arbor, MI (US)

(72) Inventors: Marc Winter, Gelnhausen (DE); Joseph B. Slater, Dexter, MI (US); James M. Tedesco, Livonia, MI (US); Carsten Bolwien, Freiburg (DE); Johannes Herbst, Freiburg (DE)

(21) Appl. No.: 18/971,413

(22) Filed: Dec. 6, 2024

(51) Int. Cl. G01N 21/65; G01N 33/00

(52) U.S. Cl. CPC G01N 21/65; G01N 33/0027

(57) ABSTRACT

A measurement device for and a method of measuring a hydrogen concentration included in a gas. The measurement device for performing the method includes: an excitation light generator transmitting excitation light to a sample of the gas; a monochromator providing a portion of Raman scattered light emanating from the illuminated sample to a measurement detector for determining a measured intensity of the portion; and an evaluation unit determining the hydrogen concentration as a linear function of the measured intensity. The portion of Raman scattered light solely includes light having wavelengths in a measurement wavelength range having a range width smaller or equal to several nanometers and including a measurement wavelength given by a wavelength at which a Raman intensity spectrum of pure hydrogen gas exhibits a maximum.

### TECHNICAL FIELD

The present disclosure relates to a measurement device for measuring a hydrogen concentration included in a gas and a method for using the same.

### BACKGROUND

Hydrogen in gas phase is an important component in many chemical processes. As an example, hydrogen is frequently used in fertilizer plants, in refineries, as well as in many other chemical plants. The importance of hydrogen will further increase because of the increasing demand for hydrogen as an energy source, replacing fossil fuels to drastically reduce carbon dioxide emissions worldwide. In order to run processes involving hydrogen in a safe and efficient manner, there is a need to measure the hydrogen concentration of gases at one or more different steps along these processes.

Hydrogen concentration measurements can, e.g., be performed with thermal conductivity detectors (TCD) used in gas chromatography to analyze inorganic gases. Thermal conductivity detectors measure the thermal conductivity of a gas between a heat source and a heat sink. The thermal conductivity of gases consisting of multiple components depends on the thermal conductivities and the concentrations of the individual components of the gas. Correspondingly, determining concentrations of individual components based on the thermal conductivity of the gas requires for the thermal conductivities of the individual components to be sufficiently different. As a result, thermal conductivity detectors exhibit a low selectivity with respect to discriminating between gas components exhibiting similar thermal properties. In addition, changes of the composition of the gas may have a negative influence on the measurement accuracy of measurements of the concentration of individual components included in the gas. Another problem is that the measurements suffer from drift and thermal interference, in particular when the thermal conductivities of individual components of the gas exhibit different temperature dependencies.

As an alternative, solid state sensors, e.g., capacitive or resistive solid state sensors, can be employed. In this case, the hydrogen concentration measurement is based on a change of an electrical property of a sensing element of the solid state sensor, e.g., a capacity or a resistivity of the sensing element, caused by an interaction of hydrogen contained in the gas with the sensing element. Solid state sensors provide a higher selectivity than thermal conductivity detectors with respect to the influence of other components included in the gas on the measurement accuracy of the hydrogen concentration. Unfortunately, solid state sensors are slow to respond to changes of the hydrogen concentration to be measured. Another problem is that solid state sensors are susceptible to contamination. As an example, sensors including palladium (Pd) to adsorb the hydrogen may be contaminated by carbon monoxide (CO) or hydrogen sulfide (H2S) adsorbing on the sensor element.

As another alternative, Raman spectroscopic measurement systems configured to analyze gases may be used to determine hydrogen concentrations included in gases. Raman spectroscopic measurement systems include a monochromatic light source illuminating a sample of the gas and a spectrograph dispersing of the Raman scattered light emanating from the sample into different wavelengths. Such systems further include a detector system receiving the dispersed Raman scattered light and determining and providing Raman intensity spectra of the Raman scattered light and an evaluation unit analyzing the Raman intensity spectra and determining the concentration of individual components of the sample based on a previously determined model. Raman spectrometric measurements systems are considerably more expensive than thermal conductivity detectors and solid state sensors. In addition, they are difficult to install. One of the reasons for this is that the model required to determine the concentrations of individual components of the gas must be adapted to the specific application where the measurement system is going to be used in a manner accounting for the impact of the background gas matrix of the gas to be analyzed on the Raman intensity spectra.

Accordingly, there remains a need for further contributions in this area of technology.

As an example, there is a need for a better method of measuring hydrogen concentrations included in gases and/or a measurement device to perform such a method, which can be manufactured at lower cost and/or is easier to install than conventional Raman spectrometric measurements systems. As another example, there is a need for a measurement device for measuring hydrogen concentrations included in gases that exhibits a shorter response time and/or is more robust, in particular less susceptible to contaminations, than solid state sensors and/or that is more selective than thermal conductivity detectors.

### SUMMARY

The present disclosure includes a measurement device for measuring a hydrogen concentration included in a gas, the measurement device including:
an excitation light generator transmitting excitation light to a sample of the gas, wherein the excitation light is configured to excite Raman scattering in the sample;
a monochromator receiving light emanating from the sample and providing a portion of Raman scattered light included in the light received by the monochromator having wavelengths in a measurement wavelength range, wherein the measurement wavelength range includes a measurement wavelength given by a wavelength at which a Raman intensity spectrum of pure hydrogen gas subjected to the excitation light provided by the excitation light generator exhibits a maximum, and wherein a range width of the measurement wavelength range is smaller or equal to several nanometers, smaller or equal to 5 nm or smaller or equal to 3 nm,
a measurement detector receiving the portion of the Raman scattered light provided by the monochromator and determining and providing a measured intensity of the portion of the Raman scattered light; and
an evaluation unit connected to the measurement detector and configured to determine the hydrogen concentration as a linear function of the measured intensity and to provide the hydrogen concentration.

The narrow measurement wavelength range including the measurement wavelength provides the advantage that the hydrogen concentration can be determined as a linear function of the measured intensity and that the contribution of other components that may be included in the gas to the measured intensity is negligibly small. The high degree of selectivity achieved by this provides the advantage that the measurement device can be put into operation without requiring a thorough analysis of the composition of the gas at the measurement site, where the measurement device is going to be used. This enables for the measurement device to be calibrated by the manufacturer and to be put into operation in a multitude of applications in a plug-and-play manner, in particular without requiring calibration measurements to be performed at the measurement site, where the measurement device is going to be employed.

Another advantage is, that measurement detectors measuring an integral intensity of incident light are available at low cost and exhibit short response times with respect to changes of the intensity of the light received by them. This provides the advantage that the measurement device exhibits short response times with respect to changes of the hydrogen concentration and that it can be manufactured at significantly lower cost than conventional Raman spectroscopic measurement systems requiring means to determine and to analyze complete Raman intensity spectra.

According to a first embodiment, the monochromator includes a filter receiving the light emanating from the sample and filtering out Raman scattered light included in the light received by the filter, wherein the filter is a single or multistage filter and/or includes a notch-filter, an edge filter, a bandpass filter and/or another type of filter element. In the first embodiment, the monochromator further includes a disperser receiving the Raman scattered light provided by the filter and dispersing the Raman scattered light into light of different wavelengths propagating in wavelength-dependent directions of propagation. The disperser is, e.g., a single or multistage disperser and/or includes a grating, a diffraction grating, a reflective grating, a holographic grating and/or another type of dispersing element, and the measurement detector is positioned such, that it selectively receives the portion of the Raman scattered light dispersed by the disperser.

In further embodiments, the measurement detector is a camera, a camera including an array of charge-coupled devices, a photodiode, a silicon photodiode, an avalanche photodiode, an avalanche silicon photodiode, or another type of detector configured to provide a measurement signal corresponding to an intensity of light received by the detector; and/or the measurement detector has an active area of 1 mm²-2 mm².

The disclosure further includes an embodiment, wherein the linear function of the measured intensity is given by a sum of a product of a proportionality factor and a difference between the measured intensity and a reference intensity and an offset, wherein the proportionality factor, the reference intensity and/or the offset is each given by a constant determined during a calibration of the measurement device, and wherein the calibration includes calibration measurements performed with the measurement device on reference samples having known hydrogen concentrations.

The disclosure further includes a second embodiment of the measurement device further comprising a reference detector connected to the evaluation unit. In the second embodiment,
the reference detector is a photodiode, a silicon photodiode, an avalanche photodiode, an avalanche silicon photodiode, or another type of detector configured to provide a measurement signal corresponding to an intensity of light received by the detector;
the reference detector is configured to determine and provide a reference intensity by determining an intensity of a portion of the Raman scattered light included in the light emanating from the sample having wavelengths in a limited reference wavelength range, wherein Raman intensity spectra of the Raman scattered light emanating from the sample exhibit a constant baseline intensity;
the evaluation unit is configured to determine the hydrogen concentration based on the measured intensity provided by the measurement detector and the reference intensity provided by the reference detector; and
the linear function of the measured intensity is a linear function of a difference between the measured intensity and the reference intensity determined by the reference detector.

In certain embodiments according to the first and the second embodiment, the reference detector is positioned such, that it selectively receives the portion of the Raman scattered light dispersed by the disperser having wavelengths in the limited reference wavelength range.

The disclosure further comprises a third embodiment, wherein the measurement device further comprises a monitoring detector configured to determine and provide a monitored intensity corresponding to an intensity of the light emanating from the sample, and wherein the monitoring detector is a photodiode, a silicon photodiode, an avalanche photodiode, an avalanche silicon photodiode, or another type of detector configured to provide a measurement signal corresponding to an intensity of light received by the detector.

In certain embodiments of the measurement device according to the third embodiment, a beam splitter is inserted between the sample and the monochromator and configured to split the incident light emanating from the sample into a main fraction transmitted to the monochromator and a minor fraction transmitted to the monitoring detector; or the monochromator includes a filter receiving the light emanating from the sample, wherein the filter is configured as a beam splitter splitting the incident light emanating from the sample into a first fraction given by Raman scattered light that is transmitted through the filter and a second fraction that is reflected by the filter to the monitoring detector.

In a further embodiment of the measurement according to the third embodiment, the monitoring detector is connected to the evaluation unit; the evaluation unit is configured to determine the hydrogen concentration based on the measured intensity provided by the measurement detector and the monitored intensity provided by the monitoring detector; the linear function is given by a sum of a product of a proportionality factor and a difference between the measured intensity and a reference intensity and an offset added to the product; the proportionality factor is inversely proportional to the intensity of the light emanating from the sample corresponding to the monitored intensity provided by the monitoring detector; and the reference intensity is either given by a constant or determined and provided by a reference detector of the measurement device configured to determine and provide the reference intensity by determining an intensity of a portion of the Raman scattered light included in the light emanating from the sample having wavelengths in a limited reference wavelength range, wherein Raman intensity spectra of the Raman scattered light emanating from the sample exhibit a constant baseline intensity.

The disclosure further includes a fourth embodiment of the measurement device further comprising an excitation detector configured to determine and provide an excitation intensity corresponding to an excitation intensity of the excitation light transmitted by the excitation light generator. In the fourth embodiment, the excitation detector is a photodiode, a silicon photodiode, an avalanche photodiode, an avalanche silicon photodiode, or another type of detector configured to provide a measurement signal corresponding to the intensity of light received by the detector. In addition or as an alternative, the excitation detector is included in the excitation light generator, or a beam splitter is inserted between the excitation light generator and the sample. In the latter case, the beam splitter is configured to split the incident excitation light into a main fraction transmitted to the sample and a minor fraction transmitted to the excitation detector.

In certain embodiments the measurement device further comprises a control unit included in or connected to the excitation light generator. These embodiments include embodiments, wherein the control unit is connected to an excitation detector determining and providing an excitation intensity corresponding to an excitation intensity of the excitation light transmitted by the excitation light generator and the control unit configured to regulate and/or to control the excitation intensity transmitted by the excitation light generator based on the excitation intensity provided by the excitation detector; and/or embodiments, wherein the control unit is connected to a monitoring detector determining and providing a monitored intensity corresponding to an intensity of the light emanating from the sample and wherein the control unit is configured to regulate and/or to control the excitation intensity transmitted by the excitation light generator based on the monitored intensity provided by the monitoring detector, and/or to increase the excitation intensity transmitted by the excitation light generator when the monitored intensity decreases and/or to decrease the excitation intensity transmitted by the excitation light generator when the monitored intensity increases.

Certain embodiments of the measurement device according to the third and the fourth embodiment further comprise a monitoring unit connected to the excitation detector and the monitoring detector. In these embodiments, the monitoring unit is configured to determine, to monitor and/or to provide a sample efficiency of the sample based on a ratio of the monitored intensity provided by the monitoring detector and the excitation intensity provided by the excitation detector, to issue a warning when the sample efficiency deviates from an initial efficiency determined at an initial time by more than a predetermined threshold value, and/or to issue an alarm when the sample efficiency drops below a predetermined minimum efficiency.

In certain embodiments, the measurement wavelength is given by the wavelength at which the Raman intensity spectrum of pure hydrogen gas subjected to the excitation light provided excitation light generator exhibits its absolute maximum; and/or the measurement-wavelength is given by a wavelength corresponding to a Raman wavenumber shift of 592 cm⁻¹.

In other embodiments, the measurement-wavelength is given by a wavelength at which the Raman intensity spectrum of pure hydrogen gas subjected to the excitation light provided excitation light generator exhibits a relative maximum or a relative maximum that is predetermined such, that a wavelength range surrounding the relative maximum, wherein contributions of multiple components that may be included in the sample in addition to the hydrogen to spectral intensities of a Raman spectrum of the sample are negligible or zero, is wider than a wavelength range surrounding the absolute maximum of the Raman intensity spectrum of pure hydrogen gas, wherein the contributions of the multiple components are negligible or zero.

In a fifth embodiment, the measurement-wavelength is given by a wavelength corresponding to a Raman wavenumber shift of 4152 cm⁻¹. In certain embodiments according to the fifth embodiment, the range width of the measurement wavelength range is 3 nm to 5 nm.

In some embodiments, the measurement device further comprises a Raman signal amplifier including an optical element focusing the excitation light onto a first focusing point in the sample and a mirror arrangement including at least one focusing mirror arranged and configured to reflect and to focus incident light onto a focusing point associated to the respective mirror such, that the excitation light and Raman scattered light resulting from interactions of the excitation light with the sample is retro-reflected into the sample at least once or multiple times before the light emanating from the sample via one of the focusing points is received and transmitted to the monochromator.

In certain embodiments, the excitation light generator includes a laser, a gas laser, a laser diode, or another type of monochromatic light source, the excitation light is monochromatic light having an excitation wavelength in a range of 250 nm to 1000 nm or in a range of 350 nm to 450 nm, the excitation light generator includes a pulsed laser and a lock-in amplifier is connected to a signal output of the measurement detector and configured to provide the measured intensity based on a measurement signal provided by the measurement detector and a reference signal provided by the excitation light generator, and/or the sample is contained in a measurement cell or a measurement cell given by a flowthrough cell including at least one transparent window enabling for the excitation light to enter the measurement cell and for the light emanating from the sample to exit the measurement cell.

The present disclosure further includes a method of measuring a hydrogen concentration included in a gas, the method comprising transmitting excitation light to a sample of the gas, wherein the excitation light is configured to excite Raman scattering in the sample and measuring a measured intensity of a portion of Raman scattered light emanating from the illuminated sample, wherein the portion of the Raman scattered light solely includes wavelengths occurring in a measurement wavelength range, a range width of the measurement wavelength range is smaller or equal to several nanometers, smaller or equal to 5 nm or smaller or equal to 3 nm, and the measurement wavelength range includes a measurement wavelength given by a wavelength corresponding to a Raman wavenumber shift of 4152 cm⁻¹, by a wavelength corresponding to a Raman wavenumber shift of 592 cm⁻¹, or by a wavelength at which a Raman intensity spectrum of pure hydrogen gas subjected to the excitation light exhibits a maximum intensity. This method further comprises determining the hydrogen concentration as a linear function of the measured intensity and providing the hydrogen concentration.

### BRIEF DESCRIPTION OF THE DRAWINGS

The described embodiments and other features, advantages and disclosures contained herein, and the manner of attaining them, will become apparent and the present disclosure will be better understood by reference to the following description of various embodiments of the present disclosure taken in junction with the accompanying drawings, wherein:

FIG. 1 shows a block diagram of a measurement device according to the present disclosure;

FIG. 2 shows an embodiment of the measurement device shown in FIG. 1;

FIG. 3 shows a further embodiment of the measurement device shown in FIG. 1, including a Raman signal amplifier;

FIG. 4 shows a further embodiment of a Raman signal amplifier;

FIG. 5 shows a representative Raman intensity spectrum of a gas;

FIG. 6 shows Raman intensity spectra of pure nitrogen, pure carbon dioxide, pure methane and pure hydrogen; and

FIG. 7 shows a method according to the present disclosure.

### DETAILED DESCRIPTION

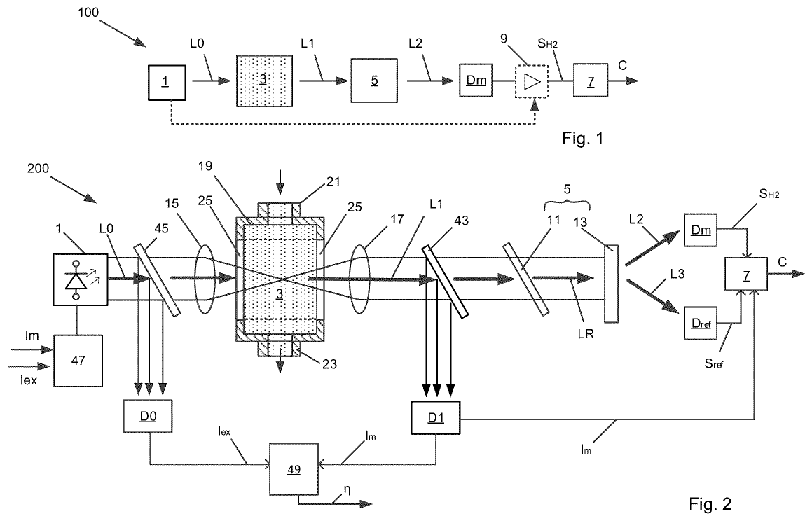

The present disclosure includes a measurement device 100 for measuring a hydrogen concentration C included in a gas. A block diagram of the measurement device 100 is shown in FIG. 1.

The measurement device 100 includes an excitation light generator 1 transmitting excitation light L0 to a sample 3 of the gas. The excitation light generator 1, e.g., includes a laser, e.g., a laser diode or a gas laser, or another type of light source providing excitation light L0 selected to excite Raman scattering in the sample 3. In at least one embodiment, the excitation light L0 is monochromatic light having an excitation wavelength λ₀ in a range of 250 nm to 1000 nm, e.g., an excitation wavelength λ₀ in a range of 350 nm to 450 nm, e.g., an excitation wavelength λ₀ of 405 nm. Monochromatic light in this range provides the advantage that the excitation wavelength λ₀ is long enough to reduce or even eliminate the excitation of fluorescence disturbing Raman spectroscopic measurements, e.g., fluorescence of contaminations of a measurement cell containing the sample 3 and/or of transparent windows of a container containing the sample 3, and short enough to ensure a high ratio of the signal strength of the Raman scattered light in relation to the excitation power.

The measurement device 100 may further include a monochromator 5 receiving light L1 emanating from the sample 3 and providing a portion L2 of the Raman scattered light included in the light L1 received by the monochromator 5 having wavelengths in a limited measurement wavelength range Δλₘ. The measurement wavelength range Δλₘ may have a range width smaller or equal to several nanometers, e.g., a range width smaller or equal to 5 nm, or a range width smaller or equal to 3 nm. In addition, the measurement wavelength range Δλₘ includes a measurement wavelength λₘ given by a wavelength at which a Raman intensity spectrum of pure hydrogen gas subjected to the excitation light L0 provided by the excitation light generator 1 exhibits a maximum.

The measurement device 100 further includes a measurement detector Dm receiving the portion L2 of the Raman scattered light provided by the monochromator 5 and determining and providing a measured intensity S_{H2} of the portion L2 of the Raman scattered light.

In certain embodiments, the measurement detector Dm may be, e.g., a camera, e.g., a camera including an array of charge-coupled devices, a photodiode, e.g., a silicon photodiode, an avalanche photodiode, e.g., an avalanche silicon photodiode, or another type of detector configured to provide a measurement signal corresponding to the intensity of light received by the detector.

The measurement wavelength range Δλₘ including the measurement wavelength λₘ is selected to ensure that the measurement detector Dm receives a significant amount of the Raman scattered light, which has been Raman scattered by hydrogen molecules included in the sample 3. At the same time, due to the relatively small width of the measurement wavelength range Δλₘ, the measurement is highly selective. This selectivity provides the advantage that the contribution of Raman scattered light that has been Raman scattered by other components that may be in the sample 3, in addition to the hydrogen, to the measured intensity S_{H2} is negligibly small. The wavelength-selective determination of the measured intensity S_{H2} of the Raman scattered light scattered by hydrogen molecules enables for the hydrogen concentration C to be determined without recording Raman intensity spectra over a wide wavelength range as generally required in conventional Raman spectroscopic analyzers and without determining an application-specific model for determining hydrogen concentration based on spectral intensities of these Raman intensity spectra at multiple different wavelengths.

The measurement device 100 may further include an evaluation unit 7 connected to the measurement detector Dm and configured to determine the hydrogen concentration C included in the sample 3 as a linear function f(S_{H2}) of the measured intensity S_{H2} determined and provided by the measurement detector Dm and to provide the thus determined concentration C.

The measurement devices of the present disclosure provide the advantages mentioned above. Individual components of the measurement device 100 may be implemented in different ways without deviating from the scope of the present disclosure. Several optional embodiments are described in more detail below.

As an example, in certain embodiments, the excitation light generator 1, e.g., includes a pulsed laser transmitting excitation light pulses. In such an embodiment, the measurement device 100 additionally includes a lock-in amplifier 9 connected to a signal output of the measurement detector Dm and providing the measured intensity S_{H2} based on a measurement signal provided by the measurement detector Dm and a reference signal provided by the excitation light generator 1. The combination of the pulsed laser with the lock-in amplifier 9 provides the advantage of an improved signal-to-noise ratio.

In addition or as an alternative, the monochromator 5 may be embodied in different ways. FIG. 2 shows an embodiment of a measurement device 200 in which the monochromator 5 includes a filter 11 and a disperser 13.

The filter 11 is configured to receive the light L1 emanating from the sample 3 along a reception path and to filter out Raman scattered light LR included in the light L1 received by the filter 11. In certain embodiments, the filter 11 may be, e.g., a single or multiple stage filter and/or include a notch-filter, an edge filter, a bandpass filter and/or another type of filter element. In certain embodiments, the filter 11 may be, e.g., a notch-filter having a filter range excluding the measurement wavelength λₘ. As an example, in combination with an excitation light generator 1 generating monochromatic excitation light LO having an excitation wavelength λ₀ of 405 nm and a measurement wavelength λₘ of 487 nm, a notch-filter having a center wavelength of 405 nm and/or a full width at half maximum of 13 nm may be employed.

The disperser 13 receives the Raman scattered light LR provided, e.g., transmitted, to the disperser 13 by the filter 11 and disperses the received Raman scattered light LR into light of different wavelengths propagating in wavelength-dependent directions of propagation. In certain embodiments, the disperser 13 may be, e.g., a single or multistage disperser and/or include a grating, e.g., a diffraction grating, a reflective grating or a holographic grating, and/or another type of dispersing element.

In FIG. 2, the measurement detector Dm is positioned such that it selectively receives the portion L2 of the dispersed Raman scattered light LR solely including wavelengths in the limited measurement wavelength range Δλₘ.

In certain embodiments, the measurement detector Dm may be a photodiode, e.g., a silicon photodiode, an avalanche photodiode, e.g., an avalanche silicon photodiode, or another type of detector having a small active area, e.g., an active area of 1 mm²-2 mm². This size of the active area provides the advantage that the wavelength range of the dispersed Raman scattered light LR received by the active area can be limited to the narrow measurement wavelength range Δλₘ simply by positioning the measurement detector Dm accordingly and without requiring a large distance between the disperser 13 and the measurement detector Dm. The latter enables the measurement device 200 to be built in a particularly compact form.

In addition or as an alternative, in certain embodiments, the measurement device 100, 200 may include at least one optical element, e.g., a mirror, a lens, a beam combiner and/or another type of optical element. FIG. 2 illustrates an example in which the optical elements include a focusing lens 15 focusing the excitation light L0 transmitted by the excitation light generator 1 onto the sample 3 and/or a collecting lens 17 collecting the light L1 emanating from the sample 3. The focusing lens 15 is disposed in the signal transmission path between the excitation light generator 1 and the sample 3. The collecting lens 17 is disposed in the signal reception path between the sample 3 and the monochromator 5.

In certain embodiments, the sample 3 may contain in a measurement cell 19. FIG. 2 shows an example in which the measurement cell 19 is a flowthrough cell, including an inlet 21 to be connected to a supply pipe providing the gas to the flowthrough cell and an outlet 23 to be connected to be a drain pipe draining the gas from the flowthrough cell. In further embodiments, the measurement cell 19 may be another type of measurement cell containing the sample 3 that enables for the excitation light L0 to be transmitted into the sample 3 and for the light L1 emanating from the sample 3 to be received.

In the embodiments shown in FIGS. 1 and 2, the excitation light generator 1 and the monochromator 5 are positioned on opposite sides of the sample 3. In such an embodiment, the measurement cell 19 includes a transparent window 25 on the side of the excitation light generator 1, through which the excitation light L0 transmitted by the excitation light generator 1 enters the measurement cell 19, and a transparent window 25 on the side of the monochromator 5 through which the light L1 emanating from the sample 3 exits the measurement cell 19.

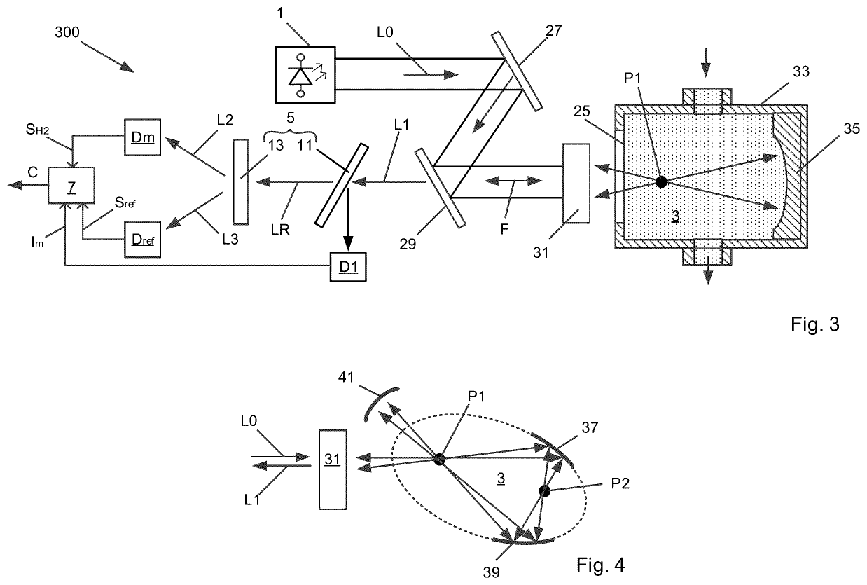

In alternative embodiments, the excitation light generator 1 and the monochromator 5 may be positioned on the same side of the sample 3. An example of a corresponding measurement device 300 is shown in FIG. 3. In such an embodiment, the excitation light generator 1 may transmit the excitation light L0 onto a mirror 27 reflecting the excitation light LO onto a beam combiner 29. The beam combiner 29 directs the excitation light L0 along a counter-propagating path F to an optical element 31 focusing the excitation light L0 onto the sample 3 and collecting the light L1 emanating from the sample 3. The beam combiner 29 is configured to transmit the light L1 emanating from the sample 3 along the counter-propagating path F to the monochromator 5, providing the portion L2 of the Raman scattered light to the measurement detector Dm. As in the embodiment shown in FIG. 2, the monochromator 5 of the measurement device 300 shown in FIG. 3 may include the filter 11 and the disperser 13. In such an embodiment, the measurement detector Dm is again positioned such that it selectively receives the portion L2 of the Raman scattered light LR having wavelength in the measurement wavelength range Δλₘ. Positioning the excitation light generator 1 and the monochromator 5 on the same side of the sample 3 provides the advantage that the sample 3 can be contained in a measurement cell 33, e.g., a flowthrough cell, including only a single transparent window 25 through which the excitation light L0 is transmitted into the sample 3 and through which the light L1 emanating from the sample 3 exists the measurement cell 33.

As another alternative, in certain embodiments, the excitation light generator 1 and the monochromator 5 may be positioned such that the monochromator 5 receives the light L1 emanating from the sample 3 along a reception path extending perpendicular to a transmission path along which the excitation light L0 is transmitted to the sample 3.

Regardless of the positioning of the excitation light generator 1 and the monochromator 5, the free propagation of the excitation light L0 and the light L1 emanating from the sample 3 provides the advantage that optical elements employed in the measurement device 100, 200, 300 can be arranged in compact manner. Examples of corresponding optical elements, e.g., include the focusing lens 15 and the collecting lens 17 shown in FIG. 2, as well as the mirror 27, the beam combiner 29 and the optical element 31 shown in FIG. 3. The free propagation further provides the advantage that a sample volume of sample 3 can be relatively small.

In certain embodiments, the measurement device 300, e.g., includes a Raman signal amplifier enhancing an excitation efficiency of the excitation light L0 exciting the Raman scattering and a collection efficiency of the collection of the resulting Raman scattered light LR. In such embodiments, Raman signal amplifiers disclosed in US 2014/0036347 A1 and in US 2008/0180663 A1, each incorporated herein by reference, may be used. Such Raman signal amplifiers include an optical element focusing the excitation light onto a first focusing point in the sample and a mirror arrangement, including at least one focusing mirror reflecting and focusing incident light, including the excitation light and Raman scattered light resulting from an interaction of the excitation light with the sample, onto a focusing point in the sample associated to the respective mirror. FIG. 3 illustrates an example in which the Raman signal amplifier includes the optical element 31 focusing the excitation light L0 onto the first focusing point P1 in the sample 3 and a mirror arrangement, including only one focusing mirror 35 in the illustrated embodiment. In this example, the focusing point associated to the focusing mirror 35 is given by the first focusing point P1 and the light L1 emanating from the sample 3 is received via the optical element 31.

FIG. 4 shows another example of a Raman signal amplifier suitable for an embodiment of the measurement device 300, wherein the optical element 31 focusses the excitation light LO onto the first focusing point P1 in the sample 3 and the mirror arrangement includes several focusing mirrors 37, 39, 41 arranged and configured to reflect and focus incident light onto focusing points P1, P2 associated to the respective mirror 37, 39, 41 such that the excitation light L0 and Raman scattered light resulting from the interaction of the excitation light L0 with the sample 3 is retro-reflected into the sample 3 multiple times before the light L1 emanating from the sample 3 via one of the focusing points P1 is received and transmitted to the monochromator 5. As an example, in FIG. 4, the light L1 emanating from the sample 3 via the first focusing point P1 is received by the optical element 31.

As mentioned above, the hydrogen concentration C may be determined as a linear function f(S_{H2}) of the measured intensity S_{H2} measured by the measurement detector Dm. As an example, in certain embodiments, the linear function f(S_{H2}) is, e.g., given by a sum of a product of a proportionality factor A and a difference between the measured intensity S_{H2} and a reference intensity S_{ref} and an offset B added to the product. In such an embodiment, the hydrogen concentration C is given by: C:=f(S_{H2})=A (S_{H2}−S_{ref})+B.

In certain embodiments, the proportionality factor A, the reference intensity S_{ref} and/or the offset B are e. g. each given by a constant. In such an embodiment, the constant values of the proportionality factor A, the reference intensity S_{ref} and/or the offset B are, e.g., determined during a calibration of the measurement device 100, 200, 300, e.g., a calibration including calibration measurements performed with the measurement device 100, 200, 300 on reference samples 3 including known concentrations of hydrogen.

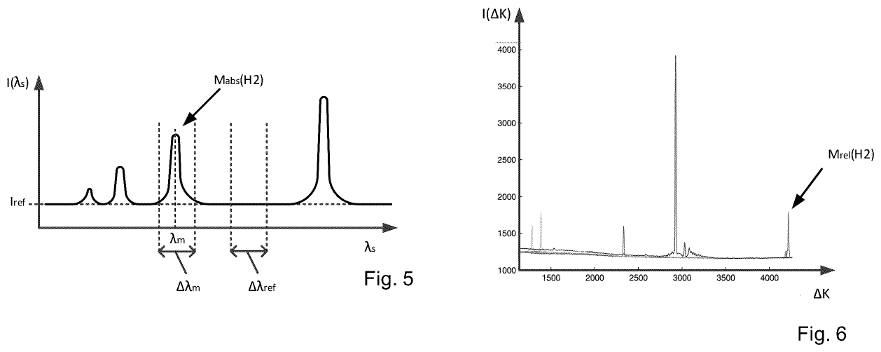

The reference intensity S_{ref}, e.g., corresponds to a base line intensity I_{ref} of the Raman spectrum of the Raman scattered light LR included in the light L1 emanating from the sample 3. This is illustrated in FIG. 5, which shows an example of a Raman intensity spectrum I(λₛ) of a gas including hydrogen and other components as a function of the wavelength λₛ of the Raman scattered light LR in a wavelength range including the measurement wavelength range Δλₘ. As shown in FIG. 5, the full Raman intensity spectrum I(λₛ) exhibits individual peaks caused by Raman scattering having maximum intensities exceeding the baseline intensity I_{ref}. Under normal operating conditions, the reference intensity S_{ref} is mainly due to noise, and can therefore be safely assumed to remain constant in a large number of applications, where the measurement device 100, 200, 300 may be employed. In certain applications, there may however be a possibility that the reference intensity S_{ref} changes during the operating time of the measurement device 100, 200, 300. To give an example, changes of the reference intensity S_{ref} may be caused by fluorescence, e.g., by fluorescence of deposits building up on the window(s) 25 of the measurement cell 19, 33.

Thus, in at least one embodiment according to the present disclosure, the measurement device 200, 300, e.g., additionally includes a reference detector Dref connected to the evaluation unit 7 and configured to determine and provide the reference intensity S_{ref}. In such an embodiment, the reference detector Dref is, e.g., configured to determine the reference intensity S_{ref} by determining an intensity of a portion L3 of the Raman scattered light LR included in the light L1 emanating from the sample 3 having wavelengths in a reference wavelength range Δλ_{ref}, in which the Raman intensity spectrum I(λₛ) of the Raman scattered light LR exhibits the constant baseline intensity I_{ref}. A corresponding example of the reference wavelength range Δλ_{ref} is shown in FIG. 5.

The width of the reference wavelength range Δλ_{ref} may be identical or at least approximately identical to the width of the measurement wavelength range Δλₘ. In this case, the reference intensity S_{ref} is, e.g., given by the intensity measured by the reference detector Dref. In certain embodiments, the reference intensity S_{ref} is, e.g., determined as a product of the intensity measured be the reference detector Dref and a factor corresponding to the ratio of the width of the reference wavelength range Δλ_{ref} and the width of the measurement wavelength range Δλₘ.

With respect to the determination of the reference intensity S_{ref} by means of the reference detector Dref, the monochromator 5 shown in FIGS. 2 and 3, including the filter 11 and the disperser 13, provides the advantage that the reference detector Dref can be positioned as shown in FIGS. 2 and 3 such that it selectively receives the portion L3 of the Raman scattered light LR dispersed by the disperser 13 having wavelengths in the limited reference wavelength range Δλ_{ref}, wherein the Raman intensity spectrum I(λₛ) of the sample 3 does not exhibit any peaks.

In certain embodiments, the reference detector Dref is, e.g., a photodiode, e.g., silicon photodiode, an avalanche photodiode, e.g., an avalanche silicon photodiode, or another type of detector configured to provide a measurement signal corresponding to the intensity of light received by the detector.

In embodiments including the reference detector Dref, the evaluation unit 7 is, e.g., configured to determine the hydrogen concentration C based on the measured intensity S_{H2} determined by the measurement detector Dm and the reference intensity S_{ref} determined by the reference detector Dref, e.g., based on a difference between the measured intensity S_{H2} and the reference intensity S_{ref}. In such an embodiment, the linear function f(S_{H2}) of the measured intensity S_{H2} is, e.g., a linear function f(S_{H2}−S_{ref}) of the difference between the measured intensity S_{H2} and the reference intensity S_{ref}. Determining the hydrogen concentration C based on this difference provides the advantage that changes of the reference intensity S_{ref} that may occur during operation of the measurement device 200, 300 are accounted for. Thus, a higher long term stability of the measurement accuracy is achieved.

The measured intensity S_{H2} not only depends on the hydrogen concentration C included in the sample 3 but also on an excitation intensity I₀ of the excitation light L0 provided by the excitation signal generator 1 and on a sample efficiency η of the sample volume of the sample 3.

In certain embodiments, the sample efficiency η is, e.g., defined as a ratio of an intensity I₁ of the light L1 emanating from the sample 3 and an excitation intensity I₀ of the excitation light L0, e.g., by η:=I₁/I₀. The dependency of the measured intensity S_{H2} on the excitation intensity I₀ of the excitation light and the sample efficiency η can be accounted for in different ways.

One approach is to assume that the excitation intensity I₀ and the sample efficiency η are both at least approximately constant. In such an embodiment, the proportionality factor A and the offset B of the linear function f(S_{H2}) employed to determine the hydrogen concentration C are both given by constants that can, e.g., be determined as outlined above. This approach provides the advantage that it can be implemented without requiring the measurement device 100, 200, 300 to include any further detection means.

Another approach is to define the proportionality factor A as a product of a constant first factor A1, a second factor A2 accounting for the sample efficiency η, and a third factor A3 accounting for the excitation intensity I₀. The second factor A2 accounting for the sample efficiency η is, e.g., defined as a ratio of an initial value of the sample efficiency η(t₀) at an initial time t₀, e.g., during calibration of the measurement device 100, 200, 300, and a current value of the sample efficiency η(t) at a current time t, e.g., by A2:=η(t₀)η(t).

The third factor A3 accounting for the excitation intensity I₀ is, e.g., defined as a ratio of an initial value of the excitation intensity I₀(t₀) at the initial time t₀ and a current value of the excitation intensity I₀(t) at the current time t, e.g., by A3:=I₀(t₀)/I₀(t).

Based on these definitions, the proportionality factor A is given by:

$$A := A 1 \cdot η ( {t}_{0} ) / η ( t ) \cdot {I}_{0} ( {t}_{0} ) / {I}_{0} ( t ) .$$

By replacing the sample efficiency η(t₀) at the initial time t₀ and the sample efficiency η(t) at the current time t by the corresponding intensity ratios given by η(t₀):=I₁(t₀)/I₀(t₀) and η(t):=I1(t)/I₀(t) the dependency of the proportionality factor A on the excitation intensity I₀ cancels out and the proportionality factor A is reduced to:

$$A := A 1 \cdot {I}_{1} ( {t}_{0} ) / {I}_{1} ( t ) .$$

Considering that the first factor A1 and the intensity I₁(t₀) of the light L1 emanating from the sample 3 at the initial time to are both constants, this representation of the proportionality factor A shows that changes of the excitation intensity I₀ and of the sample efficiency η can be simultaneously accounted for based on a single additional measurement of the intensity I₁(t) of the light L1 emanating from the sample 3 at the current time t.

In embodiments of the present disclosure in which this approach is implemented, the measurement device 200, 300 may include a monitoring detector D1 configured to determine and provide a monitored intensity Iₘ corresponding to the intensity I₁(t) of the light L1 currently emanating from the sample 3. In these embodiments, the monitoring detector D1 is connected to the evaluation unit 7, and the evaluation unit 7 is configured to determine the hydrogen concentration C based on the measured intensity S_{H2} and the monitored intensity Iₘ provided by the monitoring detector D1. In such an embodiment, the hydrogen concentration C is, e.g., determined by: C:=A1·I₁(t₀)/I₁(t)·(S_{H2}−S_{ref})+B, wherein the intensity I₁(t₀) of the light L1 currently emanating from the sample 3 is, e.g., determined as a product of a constant factor and the monitored intensity Iₘ determined and provided by the monitoring detector D1, and wherein the reference intensity S_{ref} is either given by a constant or determined and provided to the evaluation unit 7 by the reference detector Dref. Considering the initial intensity I₁(t₀) of the light L1 emanating from the sample 3 is a constant, the proportionality factor A is inversely proportional to the intensity I₁(t) of the light L1 emanating from the sample 3 corresponding to the monitored intensity Iₘ provided by the monitoring detector D1.

In certain embodiments, the monitoring detector Dm is, e.g., a photodiode, e.g., a silicon photodiode, an avalanche photodiode, e.g., an avalanche silicon photodiode, or another type of detector configured to provide a measurement signal corresponding to the intensity of light received by the detector.

FIG. 2 shows an embodiment in which a beam splitter 43 is inserted in the reception path between the sample 3 and the monochromator 5. The beam splitter 43 splits the incident light L1 into a main fraction transmitted to the monochromator 5 and a minor fraction transmitted to the monitoring detector D1 determining and providing the monitored intensity Iₘ of the light received by the monitoring detector D1.

FIG. 3 shows a further embodiment, wherein the filter 11 of the monochromator 5 is configured as a beam splitter, splitting the incident light into a first fraction including the Raman scattered light LR that is transmitted through the filter 11 to the disperser 13 and a second fraction that is reflected by the filter 11 to the monitoring detector D1. This embodiment differs from the embodiment shown in FIG. 2 in that the monitoring detector D1 does not receive the first fraction, including the Raman scattered light LR transmitted by the filter 11. This leads to a measurement error of the intensity I₁(t) of the light L1 emanating from the sample 3 of a magnitude corresponding to the intensity of the Raman scattered light LR transmitted by the filter 11. Considering that the intensity of the Raman scattered light LR emanating from the sample 3 is significantly smaller than the intensity of all other components of the light L1 emanating from the sample 3, in particular the excitation light L0 included in the light L1 emanating from the sample 3, this measurement error is negligible. At the same time, employing the filter 11 as a beam splitter provides the advantage that signal losses caused by the additional beam splitter 43 shown in FIG. 2, which also reduce the intensity of the portion L2 of the Raman scattered light LR received by the measurement detector Dm, are avoided.

Determining the hydrogen concentration C based on the monitored intensity Iₘ provides the advantage of an improved measurement accuracy of the hydrogen concentration C because changes of the excitation intensity I₀ and of the sample efficiency η that may occur during operation of the measurement device 200, 300 are accounted for. This accounting is especially advantageous in application where the sample efficiency η may vary, e. g. due to changes of the amount of excitation light L0 and/or the amount of Raman scattered light LR that is absorbed by the sample 3 and/or due to deposits building up on the window(s) 25 of the measurement cell 21, 33, which may reduce a transmissivity of the window(s) 25.

In certain embodiments, the measurement device 200 may include a control unit 47 included in or connected to the excitation light generator 1 and connected to the monitoring detector D1. The control unit 47 is, e.g., configured to regulate and/or to control the excitation intensity I₀ transmitted by the excitation light generator 1 based on the monitored intensity Iₘ determined and provided by the monitoring detector D1. In such an embodiment, the control unit 47 is, e.g., configured to increase the excitation intensity I₀ transmitted by the excitation light generator 1 when the monitored intensity Iₘ decreases and vice versa. This provides the advantage that fluctuations of the intensity I₁(t) of the light L1 emanating from the sample 3 are minimized.

In certain embodiments, the measurement device 200 may additionally include an excitation detector D0 configured to determine and provide an excitation intensity Iₑₓ corresponding to the current excitation intensity I₀(t) of the excitation light L0 transmitted by the excitation light generator 1.

FIG. 2 shows an embodiment in which a beam splitter 45 inserted in the transmission path between the excitation light generator 1 and the sample 3. The beam splitter 45 splits the incident excitation light L0 into a main fraction transmitted to the sample 3 and a minor fraction transmitted to the excitation detector D0 determining and providing the excitation intensity Iₑₓ corresponding to the intensity of the minor fraction received by the excitation detector D0. In other embodiments, the excitation detector D0 may be included in the excitation light generator 1.

Regardless of the position of the excitation detector D0, in certain embodiments, the excitation detector D0 may be a photodiode, a silicon photodiode, an avalanche photodiode, an avalanche silicon photodiode, or another type of detector configured to provide a measurement signal corresponding to the intensity of light received by the detector.

The excitation intensity Iₑₓ determined by the excitation detector D0 can be used in one or several ways. In certain embodiments, the measurement device 200, e.g., includes the control unit 47 connected to the excitation detector D0 and configured to regulate and/or control the excitation intensity I₀ transmitted by the excitation light generator 1 based on the excitation intensity Iₑₓ determined and provided by the excitation detector D0. This regulation and/or control provides the advantage that fluctuations of the excitation intensity I₀(t) of the excitation light L0 are minimized.

In embodiments including the excitation detector D0 and the monitoring detector D1, the control unit 47 may be connected to the excitation detector D0 and configured to regulate and/or control the excitation intensity I₀ based on the excitation intensity Iₑₓ as outlined above and/or connected to the monitoring detector D1 and configured to regulate and/or control the excitation intensity I₀ based on the monitored intensity Iₘ as outlined above.

In addition or as an alternative, in certain embodiments, the measurement device 200, e.g., includes a monitoring unit 49 connected to the excitation detector DO and to the monitoring detector D1. The monitoring unit 49 is, e.g., configured to determine, monitor, and/or provide the sample efficiency η of the sample 3 based on a ratio of the monitored intensity Iₘ provided by the monitoring detector D1 and the excitation intensity Iₑₓ provided by the excitation detector D0. In addition or as an alternative, the monitoring unit 49 is, e.g., configured to issue a warning when the sample efficiency n determined by the monitoring unit 49 deviates from the initial efficiency η(t₀) determined at the initial time t₀, e.g., during calibration of the measurement device 200, by more than a predetermined threshold value, and/or configured to issue an alarm, when the sample efficiency η determined by the monitoring unit 49 drops below a predetermined minimum efficiency.

Monitoring the sample efficiency η provides the advantage that changes of the sample quality, as well as impairments of the measurement cell 19, 33 containing the sample 3, e.g., fouling of the transparent window(s) 25 and/or deposits building up on the measurement cell 19, 33, affecting the sample efficiency η and thus also the hydrogen concentration measurement, will be detected at an early stage and corresponding counter measures can be taken in due time.

As mentioned above, the measurement wavelength λₘ included in the measurement wavelength range Δλₘ is given by a wavelength at which the Raman intensity spectrum of pure hydrogen gas subjected to the excitation light L0 provided by the excitation light generator 1 exhibits a maximum. Each maximum occurs at a peak position of a Raman peak included in the Raman intensity spectrum of pure hydrogen gas and is, e.g., specified by the Raman shift Δk associated to the respective peak position. Based on the definition of the Raman shift Δk being given by Δk:=(1/λ₀−1/λₛ), where λ₀ is the excitation wavelength of the excitation light L0 and λₛ is the Raman spectrum wavelength, the wavelength at which the respective maximum occurs can be calculated based on the Raman shift Δk associated to the respective maximum.

The limited width of the measurement wavelength range Δλₘ including the measurement wavelength λₘ is, e.g., predetermined based on the spectral position of the maximum employed such that a spectral overlap between the measurement wavelength range Δλₘ and spectral regions in which other components that may be included in the sample 3 of the gas exhibit Raman bands is minimized, negligible or zero.

In certain embodiments, the measurement-wavelength λₘ is, e.g., given by the wavelength at which the Raman intensity spectrum of pure hydrogen gas subjected to the excitation light L0 provided excitation light generator 1 exhibits its absolute maximum M_{abs}(H2). The absolute maximum M_{abs}(H2) occurs at a Raman wavenumber shift Δk of 592 cm⁻¹. Thus, as an example, in combination with the excitation light LO having an excitation wavelength 20 of 405 nm the measurement wavelength λₘ corresponding to the absolute maximum M_{abs}(H2) of the Raman intensity spectrum of pure hydrogen is given by 415 nm. Predetermining the measurement wavelength λₘ such that it corresponds to the Raman wavenumber shift Δk of 592 cm⁻¹ at which the Raman intensity spectrum of pure hydrogen gas exhibits its absolute maximum M_{abs}(H2) provides the advantage of correspondingly high measured intensities S_{H2}, which in turn results in a correspondingly high measurement resolution of the hydron concentration measurements.

In such embodiments, the width of the measurement-wavelength range Δλₘ is, e.g., predetermined to be relatively small, e.g., smaller or equal to 3 nm. The narrow width of the measurement wavelength range Δλₘ provides the advantage that the contribution of other components, e.g., carbon dioxide, carbon monoxide, ethane, propane, ammonia, methane, pentane, hexane, butane and/or nitrogen, that may be included in the sample 3 to the measured intensity S_{H2} measured by the measurement detector 9 is negligibly small.

As an alternative, in certain embodiments the measurement-wavelength λₘ is, e.g., given by a wavelength at which the Raman intensity spectrum of pure hydrogen gas subjected to the excitation light L0 provided by the excitation light generator 1 exhibits a relative maximum Mᵣₑₗ(H2).

In such embodiments, the relative maximum Mᵣₑₗ(H2) and correspondingly also the measurement wavelength λₘ is, e.g., predetermined such that a wavelength range surrounding the relative maximum Mᵣₑₗ(H2), in which contributions of multiple components that may be included in the sample 3, in addition to the hydrogen, to spectral intensities of a Raman intensity spectrum of the sample 3 are negligible or zero, is wider than a wavelength range surrounding the absolute maximum M_{abs}(H2), in which the contributions of the multiple components are negligible or zero. In such an embodiment, the multiple components taken into account are, e.g., given by components included in the gas at a measurement site, e.g., where the measurement device 100, 200, 300 is going to be used. As an alternative, the multiple components are, e.g., given by a predetermined selection of components frequently included in gases containing hydrogen in multiple different applications. In such an embodiment, the selection of components, e.g., includes carbon dioxide, carbon monoxide, ethane, propane, ammonia, methane, pentane, hexane, butane, and/or nitrogen.

Predetermining the measurement wavelength λₘ such that it is surrounded by a wide wavelength range in which the contributions of the multiple components are negligible or zero provides the advantage that a higher selectivity is achieved. In addition or as an alternative, based on the flexibility gained by this selection, a wider measurement wavelength range Δλₘ, e.g., a measurement wavelength range Δλₘ having a width smaller or equal to 5 nm, e.g., a width of 3 nm to 5nm is, e.g., employed. The latter provides the advantage that it increases the maximum permissible manufacturing tolerances, which in turn reduces the manufacturing costs of the measurement device 100, 200, 300.

As an example, in certain embodiments, the measurement-wavelength λₘ is, e.g., given by a wavelength corresponding to a Raman wavenumber shift Δk of 4152 cm⁻¹. The Raman wavenumber shift Δk of 4152 cm⁻¹ occurs well outside the wavenumber range normally covered by conventional Raman spectroscopic measurement systems and provides the advantage that the distance between the relative maximum Mᵣₑₗ(H2) and the nearest Raman peak associated to other components that may be included in the gas is relatively, very large. This aspect is illustrated in FIG. 6, showing Raman intensity spectra of pure nitrogen, pure carbon dioxide, pure methane and pure hydrogen in a wavenumber shift range of 1000 cm⁻¹ to 4500 cm⁻¹, wherein the relative maximum Mᵣₑₗ(H2) of the Raman intensity spectrum of pure hydrogen occurring at the Raman wavenumber shift Δk of 4152 cm⁻¹ is indicated.

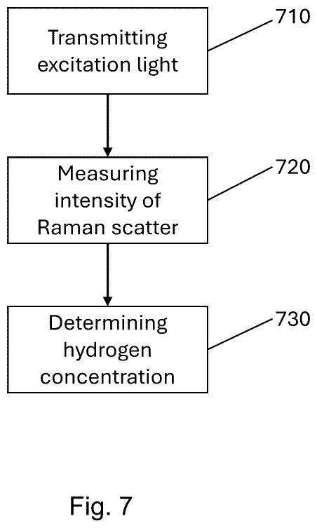

As described above in context with the measurement devices 100, 200, 300 performing hydrogen concentration measurements, the present disclosure also includes a method 700 of measuring a hydrogen concentration included in a gas, as shown in FIG. 7. The method 700 comprises a step 710 of transmitting excitation light L0 to the sample 3 of the gas and a step 720 of measuring the measured intensity S_{H2} of the portion L2 of Raman scattered emanating from the illuminated sample 3. As outlined above, the portion L2 solely includes wavelengths occurring in the measurement wavelength range Δλₘ. Again, the range width of the measurement wavelength range Δλₘ is smaller or equal to several nanometers, smaller or equal to 5 nm, or smaller or equal to 3 nm, and the measurement wavelength range Δλₘ includes the measurement wavelength λₘ given by a wavelength corresponding to a Raman wavenumber shift of 4152 cm⁻¹, by a wavelength corresponding to a Raman wavenumber shift of 592 cm⁻¹, or given by another wavelength at which the Raman intensity spectrum I(λₛ) of pure hydrogen gas subjected to the excitation light L0 exhibits a maximum intensity. The method may further include a step 730 of determining the hydrogen concentration C as a linear function f(S_{H2}) of the measured intensity and providing the hydrogen concentration as described in detail herein.

In certain embodiments, the method, e.g., further includes at least one of the further method steps performed by at least one of the measurement devices 100, 200, 300 described above with respect to the embodiments according to the present disclosure.

1. A measurement device for measuring a hydrogen concentration included in a gas, the measurement device comprising:
an excitation light generator configured to transmit excitation light to a sample of the gas, wherein the excitation light is selected to excite Raman scattering in the sample;
a monochromator configured to receive light emanating from the sample and to provide a portion of Raman scattered light included in the light received by the monochromator, the portion having wavelengths in a measurement wavelength range, wherein the measurement wavelength range includes a measurement wavelength given by a wavelength at which a Raman intensity spectrum of pure hydrogen gas subjected to the excitation light of the excitation light generator exhibits a maximum, and wherein a range width of the measurement wavelength range is smaller or equal to 5 nm or smaller or equal to 3 nm;
a measurement detector configured to receive the portion of the Raman scattered light provided by the monochromator and to determine and provide a measured intensity of the portion of the Raman scattered light; and
an evaluation unit connected to the measurement detector and configured to determine the hydrogen concentration as a linear function of the measured intensity and to provide the hydrogen concentration.

2. (canceled)

3. The measurement device according to claim 1, wherein the monochromator includes:
a filter configured to receive the light emanating from the sample and to filter Raman scattered light included in the light received by the filter, wherein the filter is a single or multistage filter and/or includes a notch-filter, an edge filter, a bandpass filter and/or another type of filter element; and
a disperser configured to receive the Raman scattered light provided by the filter and to disperse the Raman scattered light into light of different wavelengths propagating in wavelength-dependent directions of propagation, wherein the disperser is a single or multistage disperser and/or includes a grating, a diffraction grating, a reflective grating, a holographic grating and/or another type of dispersing element,
wherein the measurement detector is arranged as to selectively receive the portion of the Raman scattered light dispersed by the disperser.

4. The measurement device according to claim 1, wherein:
the measurement detector is a camera, a camera including an array of charge-coupled devices, a photodiode, a silicon photodiode, an avalanche photodiode, an avalanche silicon photodiode, or another type of detector configured to provide a measurement signal corresponding to an intensity of light received by the detector; and/or
the measurement detector includes an active area of 1-2 mm2.

5. The measurement device according to claim 1, wherein:
the linear function of the measured intensity is given by a sum of a product of a proportionality factor and a difference between the measured intensity and a reference intensity and an offset;
the proportionality factor, the reference intensity, and/or the offset are each given by a constant determined during a calibration of the measurement device; and
the calibration includes calibration measurements performed with the measurement device on reference samples having known hydrogen concentrations.

6. The measurement device according to claim 1, further comprising a reference detector connected to the evaluation unit, wherein:
the reference detector is a photodiode, a silicon photodiode, an avalanche photodiode, an avalanche silicon photodiode, or another type of detector configured to provide a measurement signal corresponding to an intensity of light received by the detector;
the reference detector is configured to determine and provide a reference intensity by determining an intensity of a portion of the Raman scattered light included in the light emanating from the sample having wavelengths in a limited reference wavelength range, wherein Raman intensity spectra of the Raman scattered light emanating from the sample exhibit a constant baseline intensity;
the evaluation unit is configured to determine the hydrogen concentration based on the measured intensity provided by the measurement detector and the reference intensity provided by the reference detector; and
the linear function of the measured intensity is a linear function of a difference between the measured intensity and the reference intensity determined by the reference detector.

7. The measurement device according to claim 6, wherein the reference detector is arranged as to selective receive the portion of the Raman scattered light dispersed by the disperser having wavelengths in the limited reference wavelength range.

8. The measurement device according to claim 1, further comprising a monitoring detector configured to determine and provide a monitored intensity corresponding to an intensity of the light emanating from the sample, wherein the monitoring detector is a photodiode, a silicon photodiode, an avalanche photodiode, an avalanche silicon photodiode, or another type of detector configured to provide a measurement signal corresponding to an intensity of light received by the detector.

9. The measurement device according to claim 8, wherein:
a beam splitter is disposed between the sample and the monochromator and configured to split incident light emanating from the sample into a main fraction transmitted to the monochromator and a minor fraction transmitted to the monitoring detector; or
the monochromator includes a filter arranged to receive the light emanating from the sample, wherein the filter is configured as a beam splitter splitting incident light emanating from the sample into a first fraction given by Raman scattered light, which is transmitted through the filter, and a second fraction, which is reflected by the filter to the monitoring detector.

10. The measurement device according to claim 8, wherein:
the monitoring detector is connected to the evaluation unit;
the evaluation unit is configured to determine the hydrogen concentration based on the measured intensity provided by the measurement detector and the monitored intensity provided by the monitoring detector;
the linear function is given by a sum of a product of a proportionality factor and a difference between the measured intensity and a reference intensity and an offset added to the product;
the proportionality factor is inversely proportional to the intensity of the light emanating from the sample corresponding to the monitored intensity provided by the monitoring detector; and
the reference intensity is either given by a constant or determined and provided by a reference detector of the measurement device configured to determine and provide the reference intensity by determining an intensity of a portion of the Raman scattered light included in the light emanating from the sample having wavelengths in a limited reference wavelength range, wherein Raman intensity spectra of the Raman scattered light emanating from the sample exhibit a constant baseline intensity.

11. The measurement device according to claim 1, further comprising an excitation detector configured to determine and provide an excitation intensity corresponding to an excitation intensity of the excitation light transmitted by the excitation light generator,
wherein the excitation detector is a photodiode, a silicon photodiode, an avalanche photodiode, an avalanche silicon photodiode, or another type of detector configured to provide a measurement signal corresponding to the intensity of light received by the detector, and/or
wherein:
the excitation detector is included in the excitation light generator; or
a beam splitter is disposed between the excitation light generator and the sample, wherein the beam splitter is configured to split incident excitation light into a main fraction transmitted to the sample and a minor fraction transmitted to the excitation detector.

12. The measurement device according to claim 1, further comprising a control unit included in or connected to the excitation light generator,
wherein the control unit is connected to an excitation detector configured to determine and provide an excitation intensity corresponding to an excitation intensity of the excitation light transmitted by the excitation light generator, the control unit further configured to regulate and/or to control the excitation intensity transmitted by the excitation light generator based on the excitation intensity provided by the excitation detector, and/or
wherein the control unit is connected to a monitoring detector configured to determine and provide a monitored intensity corresponding to an intensity of the light emanating from the sample and configured:
to regulate and/or control the excitation intensity transmitted by the excitation light generator based on the monitored intensity provided by the monitoring detector; and/or
to increase the excitation intensity transmitted by the excitation light generator when the monitored intensity decreases and/or to decrease the excitation intensity transmitted by the excitation light generator when the monitored intensity increases.

13. The measurement device according to claim 1, further comprising:
a monitoring detector configured to determine and provide a monitored intensity corresponding to an intensity of the light emanating from the sample;
an excitation detector configured to determine and provide an excitation intensity corresponding to an excitation intensity of the excitation light transmitted by the excitation light generator; and
a monitoring unit connected to the excitation detector and the monitoring detector, wherein the monitoring unit is configured to, at least one of:
determine, monitor and/or provide a sample efficiency of the sample based on a ratio of the monitored intensity provided by the monitoring detector and the excitation intensity provided by the excitation detector;
issue a warning when the sample efficiency deviates from an initial efficiency determined at an initial time by more than a predetermined threshold value; and
issue an alarm when the sample efficiency drops below a predetermined minimum efficiency.

14. The measurement device according to claim 1, wherein:
the measurement wavelength is given by the wavelength at which the Raman intensity spectrum of pure hydrogen gas subjected to the excitation light provided excitation light generator exhibits its absolute maximum; and/or
the measurement wavelength is given by a wavelength corresponding to a Raman wavenumber shift of 592 cm⁻¹.

15. The measurement device according to claim 1, wherein the measurement wavelength is given by a wavelength at which the Raman intensity spectrum of pure hydrogen gas subjected to the excitation light provided by the excitation light generator exhibits a relative maximum or a relative maximum that is predetermined such that a wavelength range surrounding the relative maximum, in which wavelength range contributions of multiple components that may be included in the sample, in addition to the hydrogen, to spectral intensities of a Raman intensity spectrum of the sample are negligible or zero, is wider than a wavelength range surrounding the absolute maximum of the Raman intensity spectrum of pure hydrogen gas, in which the contributions of the multiple components are negligible or zero.

16. The measurement device according to claim 1, wherein the measurement wavelength is given by a wavelength corresponding to a Raman wavenumber shift of 4152 cm⁻¹.

17. The measurement device according to claim 16, wherein the range width of the measurement wavelength range is 3 nm to 5 nm.

18. The measurement device according to claim 1, further comprising a Raman signal amplifier including an optical element focusing the excitation light onto a first focusing point in the sample and a mirror arrangement including at least one focusing mirror arranged and configured to reflect and focus incident light onto a focusing point associated to the respective mirror such that the excitation light and Raman scattered light resulting from interactions of the excitation light with the sample is retro-reflected into the sample at least once or multiple times before the light emanating from the sample via one of the focusing points is transmitted to and received by the monochromator.

19. The measurement device according to claim 1, wherein at least one of:
the excitation light generator includes a laser, a gas laser, a laser diode, or another type of monochromatic light source;
the excitation light is monochromatic light having an excitation wavelength in a range of 250 nm to 1000 nm or in a range of 350 nm to 450 nm;
the excitation light generator includes a pulsed laser and a lock-in amplifier, which is connected to a signal output of the measurement detector and configured to provide the measured intensity based on a measurement signal provided by the measurement detector and a reference signal provided by the excitation light generator; and
the sample is contained in a measurement cell or a flowthrough cell including at least one transparent window, which enables the excitation light to enter the measurement cell and enables the light emanating from the sample to exit the measurement cell.

20. A method of measuring a hydrogen concentration included in a gas, the method comprising:
transmitting excitation light to a sample of the gas, wherein the excitation light is configured to excite Raman scattering in the sample;
measuring a measured intensity of a portion of Raman scattered light emanating from the illuminated sample, wherein the portion solely includes wavelengths occurring in a measurement wavelength range,
wherein a range width of the measurement wavelength range is smaller or equal to several nanometers, smaller or equal to 5 nm, or smaller or equal to 3nm, wherein the measurement wavelength range includes a measurement wavelength given by a wavelength corresponding to a Raman wavenumber shift of 4152 cm⁻¹, by a wavelength corresponding to a Raman wavenumber shift of 592 cm⁻¹, or by a wavelength at which a Raman intensity spectrum of pure hydrogen gas subjected to the excitation light exhibits a maximum;
determining the hydrogen concentration as a linear function of the measured intensity; and
providing the hydrogen concentration.
